SEQUENCE LISTING (25 sequences)

SEQ ID NO: 1 - DNA, 3148 bp, Homo sapiens
gaattcccgcggagcagcgtgcgcggggccccgggagacggcggcggtagcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattc

SEQ ID NO: 2 - protein, 751 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAIPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - DNA, 2256 bp, Artificial Sequence
  C-terminal fragment of APP bearing V717F mutation
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcttcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 4 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 5 - DNA, 300 bp, Artificial Sequence
  mutant gene bCTF99(V717F)
gatgcagaattccgacatgactcaggatatgaagttcatcatcaaaaattggtgttctttgcagaagatgtgggttcaaacaaaggtgcaatcattggactcatggtgggcggtgttgtcatagcgacagtgatcttcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 6 - DNA, 26 bp, Artificial Sequence
  app-1f primer
gcagggtcgcgatgctgcccggtttg

SEQ ID NO: 7 - DNA, 24 bp, Artificial Sequence
  app-1r primer
gggggactagttctgcatctgctc

SEQ ID NO: 8 - DNA, 25 bp, Artificial Sequence
  app-2f primer
cctacaacagcagccagtacccctg

SEQ ID NO: 9 - DNA, 24 bp, Artificial Sequence
  app-2r primer
gacattctctctcggtgcttggcc

SEQ ID NO: 10 - protein, 99 aa, Artificial Sequence
  polypeptide encoded by mutant gene bCTF99(V717F)
DAEFRHDSGYEVHHQKLVFFAEDVGSNKGAIIGLMVGGVVIATVIFITLVMLKKKQYTSIHHGVVEVDAAVTPEERHLSKMQQNGYENPTYKFFEQMQN

SEQ ID NO: 11 - DNA, 24 bp, Artificial Sequence
  app-717-r primer
caaggtgatgaagatcactgtcgc

SEQ ID NO: 12 - DNA, 24 bp, Artificial Sequence
  app-717-f primer
gcgacagtgatcttcatcaccttg

SEQ ID NO: 13 - DNA, 33 bp, Artificial Sequence
  SV40pA-f primer
tccccgcggtccagacatgataagatacattga

SEQ ID NO: 14 - DNA, 32 bp, Artificial Sequence
  SV40pA-r primer
gttcgagctcataatcagccataccacatttg

SEQ ID NO: 15 - DNA, 34 bp, Artificial Sequence
  app-koz-f primer
gctctagaccatgctgcccggtttggcactgctc

SEQ ID NO: 16 - DNA, 18 bp, Artificial Sequence
  hglob-f primer
gatcctgagaacttcagg

SEQ ID NO: 17 - DNA, 18 bp, Artificial Sequence
  hglob-r primer
tctttgccaaagtgatgg

SEQ ID NO: 18 - DNA, 23 bp, Artificial Sequence
  trapp-fs primer
gcttgatatcgaattcctgcagc

SEQ ID NO: 19 - DNA, 24 bp, Artificial Sequence
  trapp-r1 primer
atgtatcttatcatgtctggaccg

SEQ ID NO: 20 - DNA, 23 bp, Artificial Sequence
  trint-f1 primer
aatgtatcatgcctctttgcacc

SEQ ID NO: 21 - DNA, 26 bp, Artificial Sequence
  app-koz-r primer
cccgcgcggcggccgcttcattaatg

SEQ ID NO: 22 - DNA, 24 bp, Artificial Sequence
  app-sig-1f primer
cgatttagatcttgacggggaaag

SEQ ID NO: 23 - DNA, 35 bp, Artificial Sequence
  app-sig-1r primer
cggaattctgcatccgcccgagccgtccaggcggc

SEQ ID NO: 24 - DNA, 20 bp, Artificial Sequence
  app99f primer
cgaattcgatgcagaattcc

SEQ ID NO: 25 - DNA, 51 bp, Artificial Sequence
  sequence encoding APP signal peptide
atgctgcccggtttggcactgctcctgctggccgcctggacggctcgggcg

(19) United States
(12) Patent Application Publication
(10) Pub. No.: US 2008/0060090 A1
(43) Pub. Date: Mar. 6, 2008

(54) Transgenic Mice Inducing Alzheimer's Disease Expressing Mutant Betactf99

(76) Inventors: Pyung Lim Han, Seoul (KR); Kang-Woo Lee, Seoul (KR); Sung-si Yang, Taejeon-si (KR); Jin-Sook Song, Taejeon-si (KR)

Correspondence Address: EDWARDS ANGELL PALMER & DODGE LLP, P.O. BOX 55874, BOSTON, MA, 02205, US

(21) Appl. No.: 10/593,672

(22) PCT Filed: Apr. 1, 2005
(86) PCT No.: PCT/KR05/00969

§ 371 Date: Jul. 20, 2007

(30) Foreign Application Priority Data
Apr. 1, 2004 (KR) 10-2004-0022562

(51) Int. Cl. A01K 67/00; C12N 15/00

(52) U.S. Cl. 800/12; 435/320.1

(57) ABSTRACT

The present invention is related to a transgenic animal inducing Alzheimer's disease. More particularly, the present invention is a vector for transformation of animal comprising a carboxyl-terminal fragments of mutant human beta amyloid protein which contains Indiana mutation (βCTF99) and a transgenic mouse inducing Alzheimer's disease prepared by microinjection of the same into a pronuclei of a fertilized oocyte. The transgenic mouse of the present invention exhibited clinical symptoms of Alzheimer's disease such as decreased of cognitive ability and memory, and increases of anxiety. Therefore, the transgenic mouse of the present invention will be useful animal model for a research of Alzheimer's disease. Particularly, since the transgenic mouse of the present invention showed more remarkable decreases of cognitive ability than any other transgenic animal model for Alzheimer's disease known in the art, the transgenic mouse of the present invention can be used as an animal model for disease relating anxiety.

### TECHNICAL FIELD

The present invention relates to a transgenic mouse with induced Alzheimer's disease pathology, more precisely, a transgenic mouse that shows Alzheimer's disease pathology induced by the insertion of a cDNA of a mutant human amyloid beta precursor protein into chromosomal DNA.

### BACKGROUND ART

Increased production of β-amyloid peptide (referred “Aβ” hereinafter) has reported to be involved in the pathogenesis of Alzheimer's disease (AD). Aβ can be produced by sequential action of β-secretase and γ-secretase inducing proteolytic cleavages of APP. Presenilin-1 (referred “PS1” hereinafter) may be a key component of γ-secretase complex or regulate traffics of its' matrix (Esler W P and Wolfe M S, 2001, Science, 293:1449-1454). Thus, PS1 has been considered to be a therapeutic target for the treatment of AD and the delayed expression of AD symptoms (Esler W P and Wolfe M S, 2001, Science, 293:1449-1454; Li Y M et al., 2000, Nature, 405:689-694). However, it is still in doubt whether or not γ-secretase inhibitor activity is involved in the accumulation of β-CTF.

Recent studies with a conditional knockout strategy have circumvented the lethality of PS1 deficient mice and generated adult animals lacking PS1 specifically in the brain (Yu H et al., 2001, Neuron, 31:713-726; Dewachter I et al., 2002, J. Neurosci, 22:3445-3453). The study involving these double transgenic mice carrying both the PS1 conditional mutation and the APP_{V717I} transgene revealed that the elimination of the γ-secretase activity provided by PS1 markedly reduced Aβ production, plaque deposition and rescued impaired hippocampal LTP, but that it neither corrected the deficits in memory that APP_{V717I} transgenic mice displayed nor stopped the progress of neurophysiological and pathogenic symptoms (Dewachter I et al., 2002, J. Neurosci, 22:3445-3453). Although the underlying mechanism has not yet been clearly elucidated, these studies show that the loss of γ-secretase activity in the brain leads to the severe accumulation of beta-CTF99, and raise the possibility that βCTF99 accumulation might cause cognitive deficits in the absence of plaque deposition in their double transgenic mice. It also raises the question as to whether or not other biochemical impairments or behavioral alterations present in APP_{V717I} transgenic mice can be reverted in their double transgenic mice. Regarding that PS1 has pleiotropic roles in brain cell functions including Norch (Naruse S et al., 1998, Neuron, 21:1213-1221; Song W et al., 1999, Proc. Natl. Acad. Sci. USA., 96:6959-6953) and N-cadherin processing (Marambaud P et al., 2003, Cell, 114:635-645), it needs to be answered whether or not the observed memory deficits of these double transgenic knockout mice were produced solely by βCTF99.

More direct evidence for the in vivo role of βCTF99 is to be ascertained from studies with transgenic mice expressing βCTF99 in the brain. Eight research groups have independently created transgenic mouse lines expressing various CTF forms of the human APP in the brain. Four of those lines showed either neuronal atrophy (Oster-Granite et al., 1996, J. Neurosci., 16:6732-6741; Nalbantoglu J et al., 1997, Science, 387:500-505; Sato et al., 1997, Dement Geriatr Cogn Disord, 8:296-307) or impaired learning (Nalbantoglu J et al., 1997, Science, 387:500-505; Berger-Sweeney J et al., 1999, Brain Res Mol Brain Res, 66:150-162; Laronde R et al., 2002, Brain Res, 956:36-44) at age 12-28 months, whereas the other four lines did not display any obvious neuronal loss or cognitive impairment (Sandhu et al., 1991, J Biol Chem., 266:21331-21334; Araki et al., 1994, Int. J. Exp. Clin. Invest., 2:100-106; Sberna et al., 1998, J. Neurochem., 71:723-731; Li et al., 1999, J. Neurochem., 72:2479-2487; Rutten et al., 2003, Neurobiol Dis., 12: 110-120). Thus, the developed transgenic mice expressing CTFs showed conflicting results that raged from no phenotype to AD-like pathogenesis. Accordingly, the in vivo role of βCTF99 remains elusive.

The present inventors have thus tried to establish an animal model for AD study, and finally prepared a transgenic mouse line bearing clinical symptoms and characteristics of AD. And further, the inventors have completed the present invention by confirming that this newly created transgenic mouse clearly shows AD symptoms.

### DISCLOSURE

### [Technical Problem]

The object of the present invention is to provide a transgenic vector that can be used to create transgenic nice showing AD pathology.

Another object of the present invention is to create a genetically stable transgenic mouse carrying the above vector.

### [Technical Solution]

To achieve the above objects, the present invention provides a transgemic vector that contains a gene coding a C-terminal fragment (CTF) of mutant human amyloid beta precursor protein (APP).

The present invention also provides a transgenic mouse that was produced by injection of the vector into the nucleus of a fertilized egg of mice, followed by transferring injected eggs into the oviduct of foster mothers to generate mice.

### [Advantageous Effects]

The transgenic mouse of the present invention showed remarkable cognitive impairments compared to those of the wild type mouse both in the Morris water maze test and in passive avoidance test. In addition, the transgenic mouse showed highly increased anxiety compared to that of the wild type mouse in the elevated plus maze test, indicating that this AD animal presents AD symptoms more clearly than any other known AD animal models. Since the transgenic mouse of the present invention shows clear AD symptoms, this animal can be used as an animal model not only for studies of AD pathogenesis but also for studies on cognitive and anxiety impairments.

### DESCRIPTION OF DRAWINGS

FIG. 1A is a schematic diagram showing the vectors ‘PDGF-βCTF99(V717F)-pA’ and ‘PDGF-intron-βCTF99 (V717F)-pA’ for transformation constructed in the present invention.

FIG. 1B is a photograph of Southern blotting confirming the insertion of βCTF99(V717F) mutant gene in transgenic animals of the invented ‘Tg-βCTF99/B6(−intron)’ and ‘Tg-βCTF99/B6(+intron)’. In the photograph, the arrow presents 350 bp βCTF99 fragment digested by SpeI.

FIG. 1C is a photograph of Northern blotting confirming the expression of βCTF99(V717F) mutant gene in transgenic mice of the invented ‘Tg-βCTF99/B6(−intron)’ and ‘Tg-βCTF99/B6(+intron)’. In the photograph, the upper arrow presents internal βCTF99 transcript (3.5 kb) and the lower arrow presents mutant βCTF99 transcript (700 pb) of the present invention.

FIG. 2A is a set of a photograph of Western blotting confirming the production of βCTF99 protein in Tg-βCTF99/B6 transgenic mice of the present invention (left panel) and a graph showing the quantification of the production above (right panel). In the photograph of Western blotting, the upper and lower panels are prepared using αCTF antibody and βCTF antibody, respectively. And in the graph, the data obtained from four different experimental groups are presented as the means±SEM.

FIG. 2B-C are photographs of immunohistological analysis investigating the expression of βCTF protein in cerebral cortex (CX) of Tg-βCTF99/B6 transgenic mouse (C) of the present invention and their wild type mice(B).

FIG. 3A is a photograph of Western blotting measuring the expressions of p-JNK, p-c-Jun, JNK1, JNK2, JNK3, p-ERK, ERK, p-p38 and p38α protein in the brain of Tg-βCTF99/B6 transgenic mouse of the present invention.

FIG. 3B is a graph showing the expression levels of p-JNK (left panel) and p-c-Jun (right panel) in the brain of Tg-βCTF99/B6 transgenic mice of the present invention. Data presented are the means±SEM of 7 and 4 independent experiments on 6 and 4 animals (n=4-6) for p-JNK and p-c-Jun, respectively.

FIG. 4A is a set of a photograph (left panel) of Western blotting measuring the expressions of Bcl-2, Bcl-x_{L}, Bad and Bax proteins in the brain of Tg-βCTF99/B6 transgenic mouse of the present invention at 14-15 months and a graph (right panel) is the results above presented in relative expression levels. Each data obtained from 3 other experimental groups is presented as the means±SEM.

FIG. 4B is a photograph resulted from immunohistological analysis of the expressions of Bad and Bax proteins in CA1, CA3 and DG regions of hippocampus (HP) in the brain of Tg-βCTF99/B6 transgenic mouse at 14-15 months of the present invention, and a graph (right panel) is the results above presented in relative expression levels. In the photograph, the scale bar in the upper panel represents 200 μm and three scale bars in the lower panel represent 500 μm each.

FIG. 5A is a photograph (left panel) of Western blotting measuring the expression of calbindin protein in the brain of Tg-βCTF99/B6 transgenic mouse at 15 months of the present invention and a graph (right panel) is the results above presented in relative expression levels. Each data obtained from three independent experimental groups is presented as the means±SEM.

FIG. 5B is a photograph of immunohistological analysis measuring the expression of calbindin protein in CA1, CA3 and DG region of hippocampus (HP) in the brain of Tg-βCTF99/B6 transgenic mouse at 15 months of the present invention, and a graph (right panel) is the results above presented in relative expression levels. The scale bar in the upper panel represents 200 μm and the scale bars in the lower panel represent 500 82 m each.

FIG. 6A is a set of a photograph (left panel) of Western blotting measuring the expressions of CREB and phosphorylated-CREB proteins in the brain of Tg-βCTF99/B6 transgenic mouse at 15 months of the present invention and a graph (right panel) is the results above presented in relative expression levels. In the graph, each data obtained from three independent experimental groups is presented as the means±SEM.

FIG. 6B-K are photographs of immunohistological analysis measuring the expressions of CREB and phosphorylated-CREB proteins in CA1 of hippocampus (HP), cerebral cortex (CX) and DG regions in the brain of Tg-βCTF99/B6 transgenic mouse at 15 months of the present invention, and a graph (right panel) is the results above presented in relative expression levels. The scale bars in panel C, E and K represent 50 μm each.

FIG. 7A-H are photographs of immunohistological analysis measuring the expressions of Neu-N protein (A-D) and MAP2 protein (E-H) in CA1 region of hippocampus (HP) and cerebral cortex (CX) of the brain of Tg-βCTF99/B6 transgenic mouse of the present invention at 18 months and the wild type control mouse.

A: Prefrontal cortex of the wild type control mouse was stained with anti-Neu-N antibody,

B: Prefrontal cortex of Tg-βCTF99/B6 transgenic mouse was stained with anti-Neu-N antibody,

C: Pyramidal cells of the wild type control mouse were stained with anti-Neu-N antibody,

D: Pyramidal cells of Tg-βCTF99/B6 transgenic mouse were stained with anti-Neu-N antibody,

E: Prefrontal cortex of the wild type control mouse was stained with anti-MAP2 antibody,

F: Prefrontal cortex of Tg-βCTF99/B6 transgenic mouse was stained with anti-MAP2 antibody,

G: CA1 of the wild type control mouse was stained with anti-MAP2 antibody,

H: CA1 of Tg-βCTF99/B6 transgenic mouse was stained with anti-MAP2 antibody.

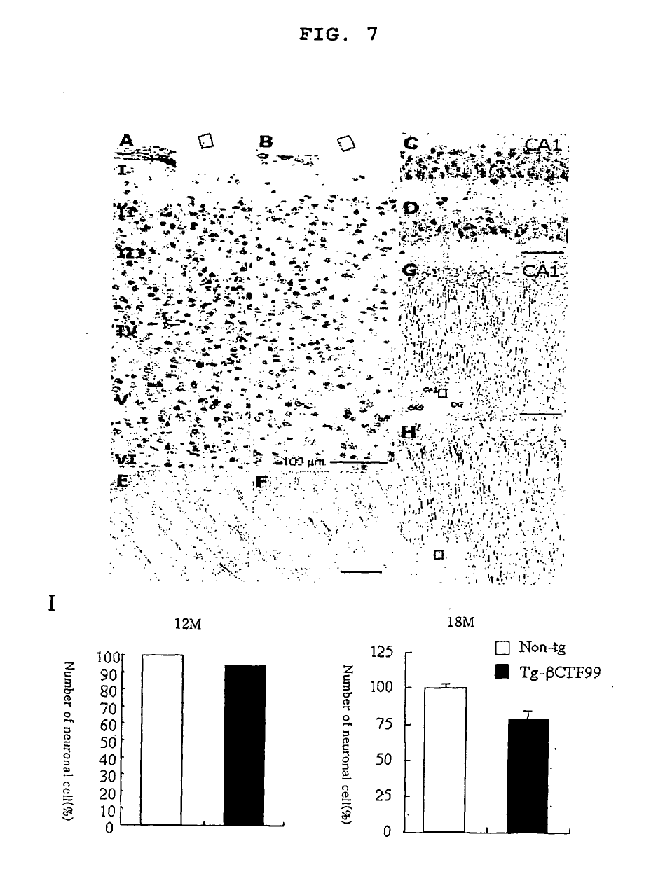

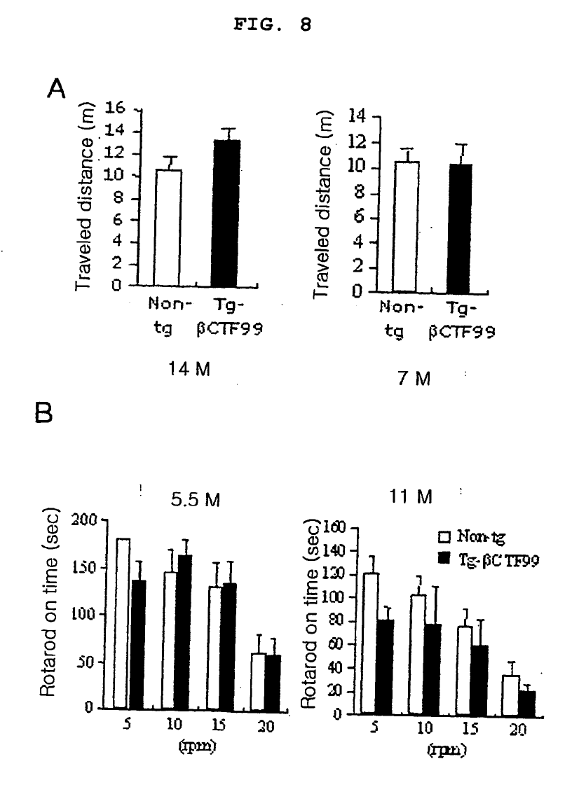

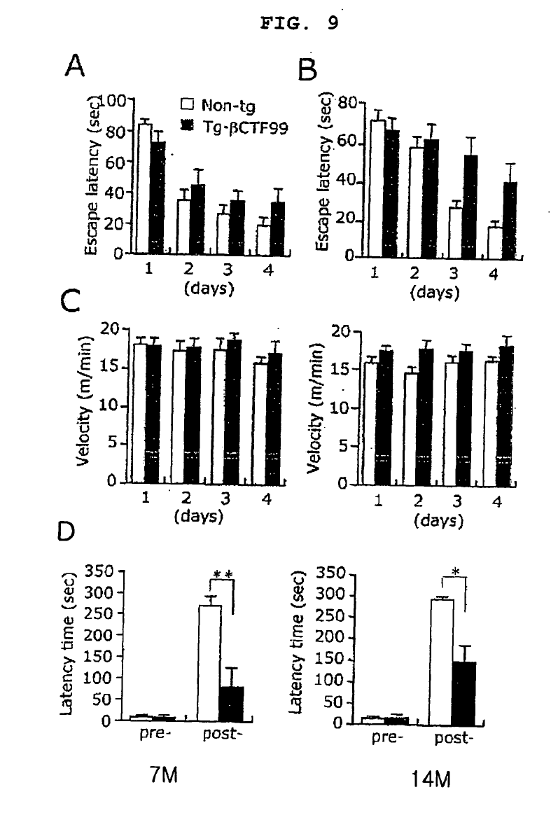

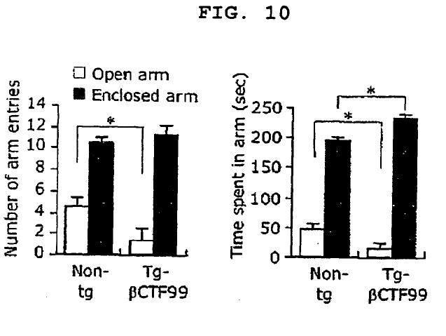

FIG. 7I is the result showing gradual neurodegeneration revealed by measuring the expression of Neu-N protein in the brains of Tg-βCTF99/B6 transgenic mice at 12 and at 18 months of the present invention.

FIG. 8A is the result of the open field test showing the locomotor activities of Tg-βCTF99/B6 transgenic mice at 7 and at 14 months of the present invention.

FIG. 8B is the result of the rota-rod test showing the locomotor activities of Tg-βCTF99/B6 transgenic mice at 5.5 and at 11 months of the present invention. In the graph, the data obtained from 6-15 independent experimental groups are shown as the means±SEM.

FIG. 9A-B is the result of Morris water maze test. The latency to find a hidden platform was recorded to investigate cognitive impairments of Tg-βCTF99/B6 transgenic mice at 7 months of age (A) and at 14 months of age (B) of the present invention. In the graph, * indicates a difference at the p<0.05 level in each group (Student's t-Lest). The data obtained from 6-8 independent experimental groups are presented as the means±SEM.

FIG. 9C shows the results of the Morris water maze test showing swimming speed of animals to find a hidden platform, which was investigated to measure whether the transgenic animals of the present invention nave any general motor function impairments. In the graph, * indicates a difference at the p<0.05 level in each group (Student's t-test). The data obtained from 6-8 independent experimental groups are presented as the means±SEM.

FIG. 9D shows the results of the passive avoidance test to investigate cognitive impairments of Tg-βCTF99/B6 mice at 7 months and at 14 months of the present invention. In the graph, * indicates a difference at the p<0.05 level in each group (Student's t-test). The data obtained from 6-8 independent experimental groups are presented as the means±SEM.

FIG. 10 shows the results of the elevated plus maze test to investigate anxiety state of Tg-βCTF99/B6 mice at 13 months of age of the present invention. In the graph, * indicates a difference at the p<0.05 level in each group (Student's t-test). The data obtained from 7-10 independent experimental groups are presented as the means±SEM.

### BEST MODE

Hereinafter, the present invention is described in detail.

The present invention provides a transgemic vector that contains a gene coding a C-terminal fragment of mutant human amyloid beta precursor protein (APP), which can be used in the generation of AD mouse model.

The above C-terminal fragment of mutant human amyloid beta precursor protein (APP) includes the C-terminal fragment of APP bearing V717F mutation, which was produced by the replacement of valine (V) with phenylalanine (F), which is prepresented by SEQ. ID. No 1. That is, the C-terminal fragment of APP bearing V717F mutation is preferred to have an amino acid sequence represented by SEQ. ID. No 3. In the preferred embodiment of the present invention, the mutant βCTF99 represented by SEQ. ID. No 3, which was then named “βCTF99(V717F)”, was prepared by PCR using the second half of APPV717F cDNA represented by SEQ. ID. No 2 as a template.

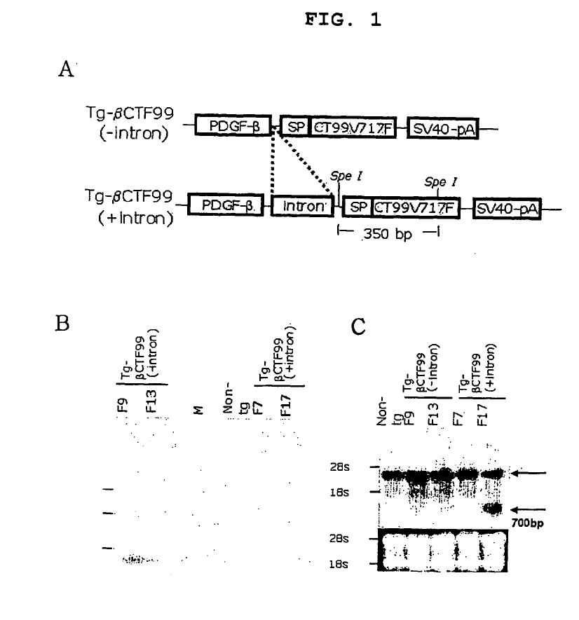

It is also preferred for the transgenic vector of the present invention, which includes PDGF-β promoter, mutant βCTF99(V717F) encoding an amino acid sequence represented by SEQ. ID. No 3, and SV40 polyadenylation sequence. To increase translate on efficiency, Kozac sequence was introduced in front of the above mutant βCTF99(V717F). The vector of the present invention was designed to include PDGF-β promoter, Kozac sequence, mutant βCTF99(V717F) represented by SEQ. ID. No 3 (βCTF99(V717F)) and SV40 polyadenylation sequence. The resulting vector was then named “PDGF-βCTF99(V717F)-polyA” (see FIG. 1).

It is also preferred to construct the transgenic vector of the present invention, which has the intron B of the human β-globin gene inserted between PDGF-β promoter and βCTF99(V717F). The introduction of the intron B gene of the human β-globin gene is to increase expression efficiency of the βCTF99(V717F) gene and transcription stability. So, in the present invention, the transgenic vector was constructed to include the PDGF-β promoter, intron B of the human β-globin gene, Kozac sequence, mutant gene coding an amino acid sequence represented by SEQ. ID. No 3 (βCTF99(V717F)) and SV40 polyadenylation sequence. The resulting vector was named “PDGF-intron-βCTF99(V717F)-polyA” (see FIG. 1A).

The present invention also provides a transgenic mouse with induced Alzheimer's disease prepared by inserting the vector of the invention into a mouse chromosome.

PDGF-βCTF99(V717F)-polyA or PDGF-intron-βCTF(V717F)-polyA transgenic vector is preferably introduced into the pronucleus of mice to produce a transgenic mouse of the present invention, and PDGF-intron-βCTF99(V717F)-polyA is more preferred. In the preferred embodiment of the present invention, PDGF-βCTF99(V717F)-polyA or PDGF-intron-βCTF99(V717F)-polyA transgenic vector was microinjected into the pronuclei of fertilized eggs prepared from inbred C75BL/6 mice, and the injected eggs were transplanted in surrogate mice. Comparison in expressions of βCTF99(V717F) mutant gene among the second generation produced from the surrogate mice and also offsprings produced by inbred was made. As a result, the expression of the mutant gene was much higher in transgenic mice transformed with PDGF-intron-βCTF99(V717F)-polyA vector than in other transgenic mice transformed with the other vector. The result indicates that it is preferred to transform a mouse by the introduction of PDGF-intron-βCTF99(V717F)-polyA vector to increase the insertion and expression efficiency of βCTF99(V717F) mutant gene of the present invention.

In the present invention, a transgenic mouse prepared by introducing PDGF-intron-βCTF99(V717F)-polyA vector for transformation into nucleus of a fertilized egg was named “Tg-βCTF/B6”. After confirming that βCTF mutant gene was successfully inserted into a mouse and so βCTF protein was expressed to the wanted level therein, the present inventors deposited the transgenic mouse of the invention at Korean Collection for Type Cultures (KCTC) of Korea Research Institute of Bioscience and Biotechnology (KRIBB) on Mar. 10, 2003 (Accession No: KCTC 10609BP).

Gradual and age-dependent decrease of the expressions of calbindin and phosphorylated-CREB protein in hippocampus of transgenic mice with Alzheimer's disease induced in them(Tg-βCTF/B6) were observed. In the meantime, neurodegeneration, motor coordination deficit, cognitive deficits and anxiety, which are characteristics shown in the brain of human AD patients, were increased.

In the preferred embodiment of the present invention, calbindin expression was significantly reduced in the hippocampus of the brain of Tg-βCTF/B6 mice at 14-15 months of age (see FIG. 5). Calbindin is one of key components of calcium-binding proteins in the brain, along with parvalbumin and calretinin, which is presented as GABAergin and pyramidal neurons in various brain regions including frontal, temporal, entorhinal and hippocampus (Mikkonen et al., 1999, Neuroscience, 92:515-532). Calcium-binding proteins regulate intracellular calcium concentrations due to their calcium buffering capacity. Altered intracellular calcium homeostasis may impair normal cellular function and potentate the cytotoxicity of neural cells (Berridge et al., 1998, Neuron, 21:13-26; Mattson, M P, 1998, Trends Neurosci, 21:53-57; Carafoli, E., 2002, Proc. Natl. Acad. Sci USA, 99:1115-1122). Mice lacking of calbindin showed impairments in spatial learning and LTP (Molinari S et al., 1996., Proc. Natl. Acad. Sci USA, 93:8028-8033). In aging or neurodegenerative brains, calbindin expression was reduced, leading to the pathologic changes (Iacopino A et al., 1990, Proc. Natl. Acad. Sci USA, 87:4078-4082; Leuba et al., 1998, Exp Neurol., 152:278-291; Bu, J. et al., 2003, Exp Neurol., 182:220-231). Although numbers of AD models have been developed as of today, no explanation has been given on the co-relation between the decrease of calbindin and the decrease of cognitive function except the recent report on transgenic mice expressing human APP mutation (Palop et al., 2003, J. Neurosci., 100:9572-9577). And the present inventors have confirmed that the decrease of calcium-binding proteins is one reason for the cognitive deficits and other deficits that are characteristic features of AD patients.

The present inventors also observed the decrease of phosphorylated-CREB expression in hippocampus region of Tg-βCTF/B6 mice (FIG. 6). Antisense oligodeoxynucleotide-mediated disruption of the CREB gene in the hippocampus was found to impair long-term memory formation (Guzoski et al., 1997, Proc. Natl. Acad. Sci USA, 94:2693-2698), and a targeted mutation or the CREBβ isoform was associated with abnormal learning and memory (Bourechuladze et al., 1994, Cell, 79:59-68; Blendy et al., 1996, EMBO J., 15:1098-1106). On the other hand, increasing the level of CREB in the brain enhanced the formation of long-term memory (Josselyn et al., 2001, J. Neurosci., 21:2404-2412). The decrease of phosphorylated-CREB expression in transgenic mice of the present invention resembles the result of investigation with AD patients. And, the reduced level of phosphorylated-CREB expression was confirmed to induce age-dependent cognitive impairment, neurodegeneration and the elevated anxiety. The above results indicate that transgenic mice of the present invention can serve as a useful AD model.

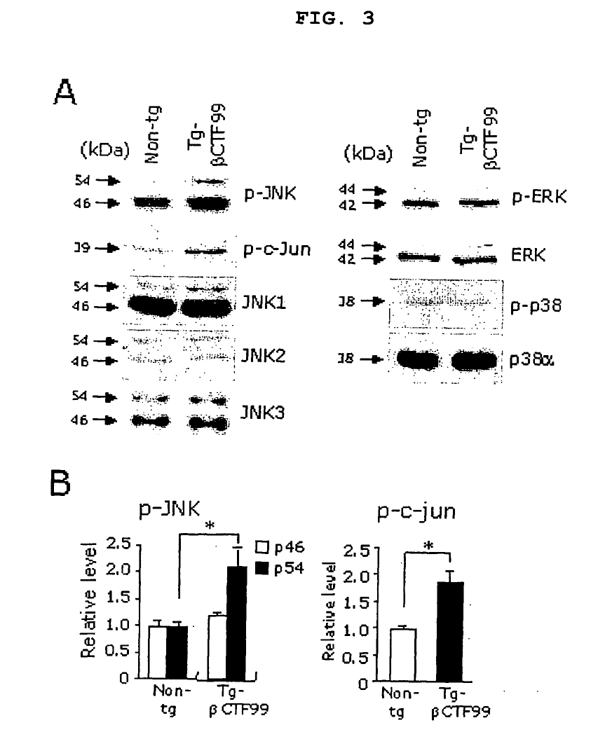

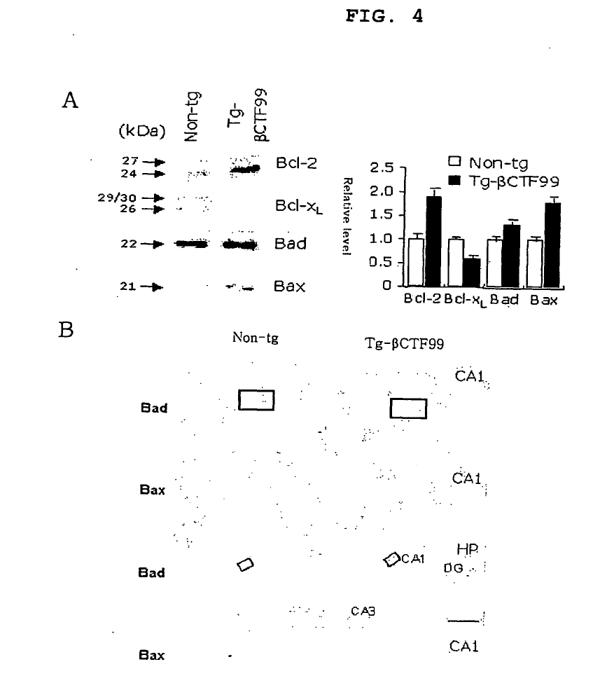

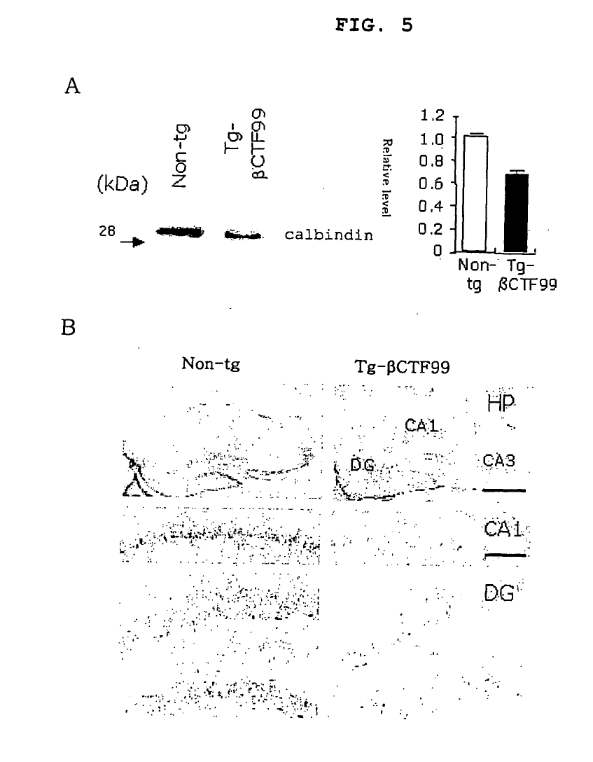

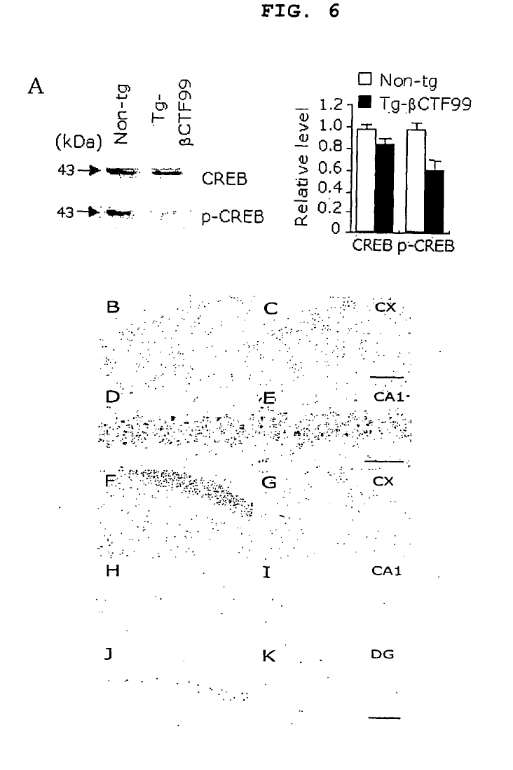

Tg-βCTF/B6 mice of the present invention showed similarities with the Tg2576+PS1P246L double transgenic mouse model (Savage et al., 2002, J. Neurosci., 22:3376-3385), as both AD models showed increased JNK activation (see FIG. 3), a feature displayed by the human AD brain (Zhu et al., 2001, J. Neurochem., 76:435-441; Savage et al., 2002, J. Neurosci., 22:3376-3385). Single transgenic mouse Tg2576 or Tg-PS1P246L did not show any changes in phosphorylated-JNK activation (Savage et al., 2002, J. Neurosci., 22:3376-3385), whereas transgenic mice of the present invention showed altered phosphorylated-JNK activation. In addition, transgenic mice of the present invention showed altered Bcl-2 family protein expressions in the brain (see FIG. 4). The expressions of Bcl-2, Bad and Bax proteins were significantly increased, whereas Bcl-x_{L} protein expression was reduced in transgenic mice of the present invention, indicating unbalanced Bcl-2 family protein expressions in the brain. The features of Bcl-2 family protein expressions in transgenic mice of the present invention were similar to those in human AD patients (Nagy et al., 1997, Neurobiol Aging, 18:565-571; Kitamura et al., 1998, Brain Res., 780: 260-269), suggesting that the transgenic mice of the present invention are very useful as an AD model.

The transgenic mice of the present invention (Tg-βCTF/B6) showed motor coordination deficit, cognitive deficits and increased anxiety, which are characteristics shown an AD patients (see FIG. 8-FIG. 10). In the preferred embodiment of the present invention, in order to confirm whether or not Tg-βCTF/B6 mice showed clinical symptoms of AD, open field test, rota-rod test, Morris water maze test and passive avoidance test were performed to investigate cognitive capacity, and elevated plus maze test was performed to investigate anxiety. As a result, in open field test, there was no significant difference in locomotor activity between wild type and transgenic mice (see FIG. 8A). In the meantime, in rota rod test, locomotor activity of transgenic mice was a little reduced, compared to that of the wild type mice, although the difference was not significant (see FIG. 8B). In Morris water maze test, transgenic mice showed cognitive deficits, resulting in impairment of memory (see FIGS. 9A-C). In passive avoidance test, transgenic mice showed impairment of memory retention, compared to that of the wild type mice (see FIG. 9D). Besides, in elevated plus maze test, notably increased anxiety was observed in transgenic mice, compared to that of the wild type mice (see FIG. 10). Thus, the transgenic mice of the present invention can be effectively used as AD models because, as described above, they showed characteristic symptoms of AD such as memory deficits, cognitive deficits and increased anxiety.

### [Mode for Invention]

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Preparation of Human Amyloid Beta Precursor Protein cDNA and βCTF99 Mutant Gene

### <1-1> Preparation of Human Amyloid Beta Precursor Protein cDNA

The cDNA coding human amyloid beta precursor protein (referred ‘APP’ hereinafter) was prepared by PCR using Marathon-Ready cDNA library (Clontech, Palo Alto, Calif., USA) constructed from human brain. The cDNA could not be amplified at once because the size of its open reading frame (ORF) was about 2.3 kb, taking APP770 as standard. Thus, the first half of the cDNA was amplified by using primer sets of app-1f primer represented by SEQ. ID. No 6 (5′-gcaagggtcqcqatgctgcccggtttg-3′, the underlined part presented Nru I restriction enzyme recognition site) and app-2r primer represented by SEQ. ID. No 9 (5′-gacattctctctcggtgcttggcc-3′), and the resulting product was digested with Nru I and Xho I. The product was inserted into Sma I and Xho I restriction enzyme recognition sites of pBluescript II KS vector (Stratagene, USA). The second half of the cDNA was amplified by using primer sets of app-2f primer represented by SEQ. ID. No 8 (5′-cctacaacagcagccagtacccctg-3′) and app-1r primer represented by SEQ. ID. No 7 (5′-gggggactagttctgcatctgctc-3′, the underlined part presented Spe I restriction enzyme recognition site), followed by digesting with Spe I and Xho T. Then, the resulting products were inserted into Spe I and Xho I restriction enzyme recognition sites of pBluescript II KS vector. The DNA fragments produced by digesting pBluescript II KS vector bearing the first half of the cDNA with BamH I and Xho I and the other DNA fragments produced by digesting pBluescript II KS vector bearing the second half of the cDNA with Xba I and Xho I were fused together with pBluescript II KS vector predigested with Xba I and BamH I, leading to the preparation of a vector construct carrying the full length of APP cDNA. In the meantime, three different isoforms were produced, according to the numbers of amino acid residues of coded protein, from the same gene of human beta amyloid by selective splicing. So, according to the numbers of amino acid residues, APP cDNA carries three different isoforms, that is, APP770, APP751 and APP695. The DNA sequences of the cloned cDNA were analyzed. As a result, among those three isoforms, the cloned cDNA was confirmed to be APP751 cDNA represented by SEQ. ID. No 1 coding APP751 (represented by SEQ. ID. No 2).

### <1-2> Preparation of APP751 Mutant Gene

“V717F mutation (Tutation in APP770 isoform Induced by the replacement of valine, the 717^{th} amino acid, with phenylalanine)” was introduced into APP751 cDNA by PCR. Particularly, PCR was performed by using pBluescript II KS vector carrying the second half of the APP751 cDNA produced in the above <Example 1-1> as a template with primer sets of app-2f primer represented by SEQ. ID. No 8 and app-717-r primer represented by SEQ. ID. No. 11 (5′-caaggtgatgaagatcactgtcgc-3′) with the 32 cycles of denaturation at 95° C. for 1 minute, primer annealing at 57° C. for 40 seconds and extension at 72° C. for 1 minute. And the resulting product was used for another PCR under the same conditions as described above by using primer sets of app717-f primer represented by SEQ. ID. No 12 (5′-gcgacagtgatcttcatcaccttg-3′) and app-1r primer represented by SEQ. ID. No 7 this time. The PCR products from the two PCR above had “V171F mutation”. So, the two PCR products were separated, slowly cooled down, extended with Klenow enzyme and then fused into one fragment. The fragment was digested with Xho I and Spe I by taking advantage of Xho I restriction enzyme recognition site of app-2f primer and Spe I restriction enzyme recognition site of app-1r-primer, which were inserted into pBluescript II KS vector which was also digested with Xho I and Spe I ahead of time and the second half of APP cDNA was inserted in, leading to the preparation of the mutant second half APP751 cDNA. In the meantime, the mutated DNA fragment prepared above was used to replace the corresponding region of pBluescript II KS vector where the full length of APP751 cDNA was inserted, resulting in APP751 mutant cDNA represented by SEQ. ID. No 3, which encodes a protein represented by SEQ. ID. No 4, and then named “hAPP(V717F)”. The nucleotide sequence of the hAPP(V717F) mutant gene was confirmed by DNA sequencing.

### <1-3> Preparation of βCTF99 Mutant Gene

The present inventors tried to prepare a protein, which includes V717F mutation in the 717^{th} amino acid region of the human amyloid beta precursor protein represented by SEQ. ID. No 1 and contains the C-terminal amino acid sequence. Particularly, the C-terminal fragment (672^{nd}-751^{st}) was amplified by PCR using cDNA (SEQ. ID. No 3) coding APP751_{v717F} protein (SEQ. ID. No 4), which induces Indiana mutation in APP protein represented by SEQ. ID. No 2, as a template, resulting in a mutant gene. PCR was performed by using the vector containing hAPP(V717F) cDNA prepared in the above <Example 1-2> as a template with primer sets of app99f primer represented by SEQ. ID. No 24 (5′-cgaattcgatgcagaattcc-3′) and appr-1r primer represented by SEQ. ID. No 7 with 32 cycles of denaturation at 95° C. for 1 minute, primer annealing at 57° C. for 40 seconds and extension at 72° C. for 1 minute. The PCR product was digested with EcoR I and Spe I, which was linked to pBluscriptE KS vector (Stratagene) pre-digested with EcoR I and Spe I. Thus, the C-terminal of APP bean to carry mutation, and the mutant gene was represented by SEQ. ID. No 5 and coded a protein represented by SEQ. ID. No 10. The resulting mutant gene was then named “βCTF99”. The nucleot de sequence of the mutant gene was confirmed by DNA sequencing.

In order to insert signal peptide in the above mutant gene, signal peptide region was amplified by PCR using pKS-aap696-1/2 vector bearing signal peptide as a template. The PCR was performed by using primer sets of app-sig-1f primer, represented by SEQ. ID. No 22, having Bgl II recognition site and app-sig-1r primer, represented by-SEQ. ID. No 23, having EcoR I recognition site, with 32 cycles of denaturation at 95° C. for 1 minute, primer annealing at 55° C. for 1 minute and extension at 72° C. for 1 minute. The PCR product was digested with Bgl II, linearized by Kienow enzyme, digested with EcoR I, and then subclcned into EcoR I digesting region of pBluescript II KS vector (Stratagene) digested with BamH I, linearized by Klenow enzyme and digested with EcoR I.

In order to enhance translation efficiency of the signal peptide, PCR was performed by using pBluescript II KS vector harboring the signal peptide as a template with primer sets of app-koz-f primer represented by SEQ. ID. No 15 having Xba I recognition site and Kozac sequence (GACC) and app-koz-r primer represented by SEQ. ID. No 21 having Nvot I recognition site, followed by insertion of Kozac sequence (GACC) in front of starting codon (ATG) of the signal peptide. The PCR product was digested with Xba I and Not I, which was fused into pBluescript II KS digested with Xba T and Not I, resulting in the construction of pKS-kozappsig vector. The vector was digested with EcoRI and the constructed βCTF99 vector was digested with EcoRI. The both digested products were fused, leading to the preparation of a vector producing a fusion protein where signal peptide and βCTF99 were fused. So, the recombinant protein containing Kozac sequence, signal peptide and a gene coding βCTF99 protein in which Indiana mutation was induced was prepared and named “βCTF99(V717F)”.

### EXAMPLE 2

### Construction of an Expression Cassette Containing βCTE99(V717F) Mutant Gene for Transgenic Animal

In order to prepare an AD animal model, an expression cassette for transformation containing βCTF99(V717F) mutant gene was constructed. Particularly, pGK-neo-PA vector (Lee et al., J. Neurosci., 2002, 15:7931-7940) was amplified using primer sets of SV40pA-f primer represented by SEQ. ID. No 13 (5′-tccccqcqgtccagacatgataagatacattga-3′, the underlined part presented Sac II restriction enzyme recognition site) and SV40pA-r primer represented by SEQ. ID. No 14 (5′-gttcqaqctcataatcagccataccacatttg-3′, the underlined part presented Sac I restriction enzyme recognition site), resulting in 247 bp sized SV40-pA fragment for polyadenylation signal of the mutant gene. Then, the fragment was digested with Sac II and Sac I, which was inserted into pBluescript II KS vector. PsisCAT6a vector (Sasahara, M. et al., Cell, 1991, 64(1):217-27) was digested with Xba I, linearized with Klenow enzyme, and digested with Hind III, resulting in human platelet-derived growth factor-beta (PDGF-beta) promoter fragment. The obtained PDGF-beta promoter fragment was inserted into pBluescript II KS vector that was digested with Sal I, linearized with Klenow enzyme and then digested again with Hind III. The pBluescript II KS vector bearing the above PDGF-beta promoter fragment was digested with Kpn I and Hind III, resulting in 1.5 kb sized PDGF-beta promoter fragment. And the fragment was inserted into Kpn I and Hind III recognition sites of pBlescript II KS vector containing SV40 pA region. The resulting vector, thus, has a structure that has PDGF-beta promoter and SV40-pA region respectively at each side of multiclonlng site of pBluescript II KS vector. In the vector, βCTF99(V717F) mu-ant gene containing Kozac sequence (GACC) in front of starting codon, prepared in the above <Example 1>, was inserted, resulting in an expression cassette for transformation. Finally, the expression cassette was constructed to possess PDGF-βpromoter-βCTF99(V717F)-pA in that order, and named “PDGF-βCTF99(V717F)-pA” (FIG. 1A).

### EXAMPLE 3

### Construction of an Expression Cassette Containing Intron and βCTF99(V717F) Mutant Gene for Transgenic Animal

The intron elevates expression efficiency of a mutant gene and increases transcription stability. Thus, in order to introduce a mutant gene into an animal model effectively, the present inventors introduced the intron B gene (918 bp) (Choi et al., Molecular and cellular biology, June 1991, p. 3070-3074; Palmiter et al., PNAS, 1991, 88:478-482) of human β-globin gene into the expression cassette prepared in the above <Example 2>. Precisely, the intron B of human β globin gene was amplified by PCR using the primers of hglob-f represented by SEQ. ID. No 16 and hglob-r represented by SEQ. ID. No 17 and genomic DNA, which was obtained from the human neuroblastoma cell line SH-SY5Y, as a template. The amplified intron B gene product derived from human β-globin gene was sub-cloned into pGEM-T Easy vector (Promega, Madison, Wis., USA), which was inserted between PDGF-β promoter gene of PDGF-βCTF99(V717F)-pA expression cassette constructed in the above <Example 2> and βCTF99(V717F) mutant gene. The resulting expression vector for transformation was named “PDGF-βCTF99(V717F)-pA” (FIG. 1A).

### EXAMPLE 4

### Generation of Transgenic Animals

PDGF-βCTF99(V717F)-pA expression cassette constructed in the above <Example 2> and PDGF-intron-βCTF99(V717F)-pA expression cassette constructed in the above <Example 3> were digested with a restriction enzyme (BssHII), resulting in 3.1 kb sized linearized fragment. The product was microinjected into the pronuclei of fertilized eggs prepared from inbred C57BL/6 mice. After the microinjection, the fertilized eggs were transferred to the oviduct of pseudopregnant female (ICR) mice. The methods for transformation of animals used in the present invention including microinjection were in accordance with the conventional methods (Games et al., Nature, 1995; Hisao et al., Science, 1996).

### EXAMPLE 5

### Confirmation of the Insertion of a Mutant Gene into Chromosomal DNA

Genomic DNA was extracted from the tails of F1 mice generated from the animal transformation procedure performed in the above <Example 4>, and PCR was performed to confirm whether or not a mutant gene was rightly inserted into nuclei of fertilized eggs. Particularly, PCR was performed with primer sets of trapp-fs primer represented by SEQ. ID. No 18 and trapp-r1 primer represented by SEQ. ID. No 19, in order to investigate the insertion of a mutant gene into the F1 mice generated by using PDGF-βCTF99(V717F)-pA expression cassette excluding intron. In the meantime, another PCR was performed with primer sets of trint-f1 primer represented by SEQ. ID. No 20 and sv40pA-r primer represented by SEQ. ID. No 14, in order to investigate the insertion of the mutant gene in the F1 mice generated by using an expression cassette including intron (PDGF-intron-βCTF99(V717F)-pA).

As a result, among the F1 mice generated by the introduction of an expression cassette excluding intron (PDGF-βCTF99(V717F)-pA), 16 mice were confirmed to bear the expression cassette. In the case of F1 mice generated by the introduction of an expression cassette including Intron (PDGF-intron-βCTF99(V717F)-pA), only 2 mice were confirmed to bear the expression cassette.

Southern blot analysis was also performed to confirm the introduction of the expression cassette of the present invention. Precisely, genomic DNA was extracted from the tails of F1 mice generated from the animal transformation procedure taken in the above <Example 4>, and then 15 μg of the genomic DNA was digested with restriction enzyme Spe I. The resulting products were electrophorezed on agarose gel, and then transferred onto nitrocellulose membrane. Hybridization was performed using a ³²P-labeled probe prepared from the 350 bp SpeI fragment at the C-terminus of APP cDNA, and the results were developed on X-ray film.

The results resembled those of the above PCR with the genomic DNA, that is, among the F1 mice generated by the introduction of an expression cassette excluding intron (PDGF-βCTF99(V717F)-pA), 16 mice were confirmed to bear the expression cassette. In the case of those F1 mice generated by the introduction of an expression cassette including intron (PDGF-intron-βCTF99(V717F) -pA), only 2 mice were confirmed to bear the expression cassette (FIG. 1B).

The transgenic mice which were confirmed by genoxic PCR and Southern blotting, to bear the βCTF(V717F) mutant gene of the present invention were inbred with C57BL/6 mice.

### EXAMPLE 6

### Investigation of the Transgene Expression

In order to confirm whether or not βCTF99(V717F) mutant gene was successfully introduced and expressed in the transgenic mice of the present invention, total RNA was prepared from the brains of transgenic mice, followed by Northern blotting. Northern blot analysis was performed according to the method of Lee, et al. (Lee et al., J Neurosci, 2002, 15:7931-7940). Precisely, total RNA was prepared from the brains of wild type and transgenic mice at 2 months, which were confirmed to have βCTF99(V717F) mutant gene transducted in the above <Example 4> and <Example 5>. Trizol reagent (Sigma, St. Louis, Mo., USA) was used for the extraction of the total RNA. A membrane blot carrying 30 μg of total RNA was prepared after separating on denaturing agarose gel (1% agarose, 6.2% formaldehyde in 1× MOPS), and hybridized with a ³²P-labeled probe prepared from the SpeI-digested fragment (350 bp) of CTF99, which was also used in the above <Example 5> for Southern blot analysis, and then the results were developed on X-ray film. The probe was able to recognize both internal APP transcript and βCTF99(V717F) mutant transcript.

As a result, the expression of APP mutant transcript was much higher as a whole than that of the endogenous APP transcript in transgenic mice, regardless of that βCTF99(V717F) mutant gene transforming a mouse included intron or not. In particular, the expression of βCTF99(V717F) was especially higher in transgenic mice (F17 mice) bearing βCTF99(V717F) mutant gene harboring intron (FIG. 1C). And those transgenic mice showing the high expression of βCTF99(V717F) mutant gene including intron were named “Tg-βCTF99/B6”, which were, from then on, used for further experiments of the present invention.

The transgenic mouse Tg-βCTF99/B6 was deposited at Korean Collection for Type Cultures (KCTC) of Korea Research Institute of Bioscience and Biotechnology (KRIBB) on Mar. 10, 2003 (Accession No: KCTC 10609BP).

### EXAMPLE 7

### Protein Production by the Transgene

It was confirmed in the above <Example 6> that Tg-βCTF99/B6 mice of the present invention expressed βCTF99(V717F) mutant gene successfully. In order to investigate the possibility of protein production by the expressed gene, total protein was extracted from the brains of wild type controls and Tg-βCTF99/B6 mice at 4-5 months, followed by Western blotting. The Western blot analysis was performed according to the method of Lee, et al. (Lee et al., Brain Res Mol Brain Res, 1999, 70:116-124). Particularly, mouse brain tissue was nomogenized in 4° C. lysis buffer (50 mM Tris-HCl, pH 8.0, 150 mM NaCl, 1% NP-40, 0.1% SDS, 0.5% sodium deoxycholate) containing 1 mM phenylmethylsulfonyl fluoride, and protease inhibitor (Complete™; Roche, Mannheim, Germany). Centrifugation was performed with the homogenized brain tissue samples at 13,000 rpm, for 20 minutes at 4° C. to obtain supernatant. The protein in the supernatant was quantified by BCA quantification kit (Sigma, St. Louis, Mo., USA). Each lane was loaded with 30 μg of the protein, and acrylamide gel electrophoresis was carried out. The separated proteins were transferred onto a PVDF membrane (Bio-Rad, Hercules, Calif., USA) and The membranes were blocked with 5% non-fat dry milk, 2% BSA, 4% FBS, 4% horse serum, 4% goat serum in Tris-buffered saline and 0.1% Tween 20. Two βCTF-specific polyclonal antibodies commonly detected the endogenous ˜12 kD βCTF99 (A8717; Sigma, St. Louis Mo., USA) and −10 kD αCTF83(p3) (51-2700; Zymed, San Francisco, Calif., USA) fragments were used for the Western blot analysis to confirm the production of the βCTF99(V717F) mutant protein. The βCTF99 and αCTF83 are proteins generated by β-secretase and α-secretase, respectively, in the brains of non-transgenic controls. Immunoblots were detected using ECL detecting reagents (Santa Cruz, Calif., USA).

For the detection of βCTF99, the brain tissues were homogenized in 1:10 (g/vol) Tris-buffered saline (TBS) containing 50 mM Tris-HCl (pH 8.0), 175 mM NaCl, 5 mM EDTA, 2 mM phenylmethylsulfonyl fluoride, and a protease inhibitor cocktail (Complete™; Roche, Mannheim, Germany). Fifty μg of the protein sample were mixed with an equal volume of 2> Laemmli sample buffer containing 10% β-mercaptoethanol, boiled for 10 min, and then electrophoresed on 16.5% Tris/tricine agarose gel as described (Li et al., 1999). After being transferred onto PVDF membranes, the resolved proteins were probed with polyclonal anti-CTF. Immunoblots were detected using ECL detection reagents.

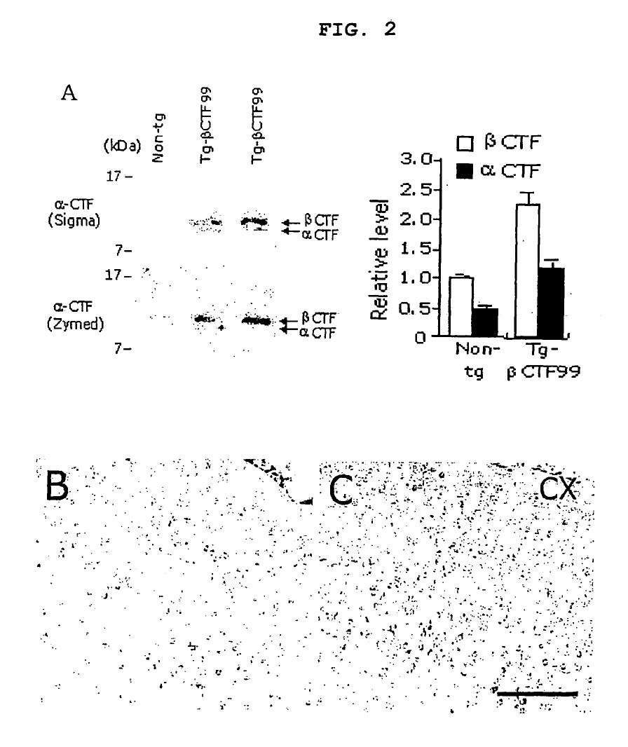

As a result, in the Tg-βCTF99/B6 brain at 4-5 months, the amounts of βCTF99 and αCTF83 were notably up-regulated. Densitometric measurements of the CTFs using computer-assisted imaging software indicated that the expression levels of βCTF99 and αCTF83 were 2.63±0.37 and 2.61±0.2 fold that of endogenous βCTF99 and αCTF83 (FIG. 2A).

### EXAMPLE 8

### Immunohistochemical Analysis of the Brains of Transgenic Mice

For immunohistochemical experiments, the mice were perfused with 0.9% saline through ascending aorta, and then perfused again with 4% paraformaldehyde in 0.1 M phosphate buffer (referred “PB” hereinafter, pH 7.4). The brain was removed and fixed in the fixative at 4° C. The fixed brain was carnally cut into 40 μm-thick sections with a vibratome. The sections were reacted in 3% hydrogen peroxide solution dissolved in 0.1 M PB (pH 7.4) for 30 minutes and washed with PB. The sections were blocked by 5% normal goat serum, 2% BSA and 2% FBS for 2 hours at room temperature. The primary antibody was added to the blocking buffer, which was left at 4° C. for overnight for reaction. After washing with PB solution, the secondary antibody, which was biotinylated by being diluted 1:200 fold, was added. Then, 1:100 fold diluted avidin and biotinylated HRP complex (Vector Laboratories, Burlingame, Calif.) were also added for one more hour reaction. 0.05% 3,3′-diaminobenzidine and 0.001% hydrogen peroxide in 0.1 M Tris (pH 7.4) were used for the color development. Cerebral cortex (referred “CX” hereinafter), pyramidal cells of CA1-CA3 regions (referred “CA1”-“CA3” hereinafter), hippocampus (referred “HP” hereinafter) and dentate gyrus (referred “DG” hereinafter) were used for the analysis.

As a result, increased expression of βCTF99 protein was observed in neuronal cells of broad brain regions including cerebral cortex in Tg-βCTF/B6 mice of the present invention (FIG. 2B-C). However, plaque like-Aβ deposition was not found in the brains of Tg-βCTF/B6 mice at up to 18 months. And, approximately 92% of the Tg-βCTF/B6 mice survived until at least 480 days, suggesting that those transgenic mice can survive longer than the conventional transgenic mice, even though the lethality is elevated compared to that of the wild type control mice. Thus, the transgenic mice of the present invention are much effective as animal models.

### EXAMPLE 9

### Expressions of Other Proteins in the Brains of Transgenic Mice

Expressions of other proteins that might be affected by βCTF99(V717F) mutant gene introduced into Tg-βCTF99/B6 mice of the present invention were investigated. Particularly, Western blot analysis was performed with brain tissues by the same way as described earlier in the <Example 7>. The antibodies used for the analysis were anti-phospho-JNK antibody (9251S; Cell Signaling, Beverly, Mass., USA), anti-phospho-c-Jun antibody (9261S; Cell Signaling), anti-phosohc-p38 antibody. (9211S; Cell signaling), anti-JNK3 antibody (06-749; Upstate Biotechnology, Lake placid, N.Y., USA), anti-CREB antibody (Upstate Biotechnology), anti-phospho-CREB antibody (Upstate Biotechnology), anti-MAP2 antibody (Upstate Biotechnology), anti-calbindin antibody (C9848; Sigma, St. Louis, Mo., USA), anti-parvalbumin (P3088; Sigma), anti-calretinin antibody (AB5054; Chemi-Con, Temecula, Calif., USA), anti-JNK1 antibody (15701A; Pharmingen, San Diego, Calif., USA), anti-JNK2 antibody (sc-572; Santa Cruz Bio-Technology, Santa Cruz, Calif., USA), anti-phospho-ERK antibody (sc-7383; Santa Cruz Bio-Technology), anti-ERK antibody (sc-154; Santa Cruz Bio-Technology), anti-Bcl-2 antibody (sc-783; Santa Cruz Bio-Technology), anti-Bad antibody (sc-942-G), anti-Bax antibody (sc-6236; Santa Cruz Bio-Technology), and anti-Bcl-xL (sc-7195; Santa Cruz Bio-Technology).

Recent reports indicate that the human AD brain shows phospho-JNK up-regulation (Zhu et al., 2001, J Neurochem., 76:435-441; Savage et al., 2002, J Neurosci., 22: 3376-3385). Thus, the present inventors performed Western blot analysis to examine the expression of phospho-JNK protein in the brain of the transgenic mouse of the present invention. As a result, the expressions of phospho-JNK protein and phospho-c-Jun protein in the brain of Tg-βCTF99/B6 at 15 months were higher than those of age-matched wild type controls, whereas the expressions of JNK1, JNK2, JNK3, phospho-ERK and phospho-p38 were not significantly changed (FIG. 3).

Bcl-2 and Bcl-xL proteins are anti-apostolic whereas Bax and Bad proteins are pro-apoptotic. And these are B-cell leukemia-2 (Bcl-2) family proteins (Davies et al., 1993, Trend Neurosci., 18:355-358). The present inventors also performed Western blot analysis to detect the change of expression level of Bcl-2 family protein in the brain of the transgenic mice. As a result, the expressions of Bcl-2, Bad and Bax were significantly elevated, whereas Bcl-2-xL expression was attenuated in Tg-βCTF99/B6 brain at 14-16 months (FIG. 4A). The result indicates that the expression of Bcl protein is affected by the insertion of βCTF99(Ld) mutant gene of the present invention. Based on the result, immunohistochemical analysis was performed to examine the expressions of Bad and Bax in CA1 region, the pyramidal cell layer of the hippocampus, in analogy to the procedure as described in the <Example 8>. Consistent with the results of Western blot analysis above, the expressions of Bad and Bax proteins in CA1 region were increased (FIG. 4B).

It has been known that AD brain shows the increased expression of calcium-binding proteins (Anthony et al., 1990, Proc. Natl. Acad. Sci USA., 87:4078-4082; Mikkonen et al., 1999, Neuroscience, 92:515-532; Bu et al., 2003, Exp Neurol., 182:220-231). Thus, the expressions of calcium-binding proteins such as calbindin, parvalbumin and calretinin were investigated by Western blot and immunohistochemical assay. As a result, calbindin expression was reduced in hippocampus, CA1, CA3 and DG regions of Tg-βCTF99/B6 at 14-16 months, compared to that of the wild type controls (FIG. 5A—Western blot, FIG. 5B—Immunohistochemical assay). Calbindin expression was not detected in the brains of Tg-βCTF99/B6 at 4-5 months. In the meantime, parvalbumin and calretinin expressions were not much different from those of wild type controls.

Recent reports indicate that the phospho-CREB level is reduced in the AD brain, which is nothing to do with the variations of total CREB protein level, though (Yarqamoto-Sasaki et al., 1999, J Neurosci., 22:1858-1867). Especially, the increased expression of CREB protein during neuronal activity induces synaptic plasticity, in particular hippocampus-based memory retention (Mayford et al., 1999, Trends Genet., 15:463-470; Colombo et al., 2003, J Neurosci., 23:3547-3554; Viola et al., 2000, J Neurosci., 20: RC112 (1-5)). Accordingly, the present inventors performed Western blot and immunohistochemical analysis to examine phosohc-CREB protein expression at transgenic mice of the present invention. As a result, total CREB protein expression was not changed in the brain of Tg-βCTF99/B6 at 14-16 months, whereas phospho-CREB protein expression was reduced in hippocampus, CA1 and CX regions of the transgenic mice, compared to that of the wild type controls (FIG. 6A—Western blot, FIG. 6B—K-Immunohistochemical analysis). However, phospho-CREB protein expression in the brain of Tg-βCTF99/B6 at 5-7 months was similar to that of wild type controls.

In order to examine the possibility of neuronal loss by βCTF99(V717F) mutant gene of the present invention, the expression of neuron-specific marker MAP-2 protein was measured. As a result, the expression of MAP-2 protein was reduced in CX and hippocampus CA1 region of the brain of Tg-βCTF99/B6 at 15-18 months, indicating that the mutant gene had influence on neuronal formation (FIG. 7A-H). However, the level of the protein in the brain of the transgenic mouse at 7 months was not much different from that of a wild type control.

In order to examine the possibility of neuronal degeneration by βCTF99(Ld) mutant gene of the present invention, the expression of Neu protein was investigated. As a result, neuronal cell density was approximately 5-10% reduced at the transgenic mice at 11-12 months, which went further to 25% reduction at 18 months. The result indicates that βCTF99(Ld) mutant gene of the present invention induces gradual neuronal degeneration (FIG. 7I).

### EXAMPLE 10

### Cognitive Function of the Transgenic Mice

Histopathological characteristics of AD brain are (1) the deposition of extracellular senile plaques, (2) the formation of intracellular neurofibrillary tangle, (3) the degeneration of axons and synapses, and neuronal loss, and (4) malfunction of the brain by neuronal loss, which are all detectable by histological test. In particular, cognitive deficits are the most characteristic and important morphological and clinical symptom. Thus, it is important for an AD animal model to show not only histological characteristics including senile plaques deposition, but also, in fact more importantly, cognitive deficits. The present inventors performed Morris water maze test, passive avoidance test and open field test to judge the cognitive deficits in candidates for AD models.

Mice were housed in cages in a temperature- and humidity-controlled environment with a 12 hour-light/dark cycle (light switched on at 7 a.m.). All animals were handled in accordance with the animal care guideline of Ewha Womans University School of Medicine. To track the animals' behavior, a computerized video-tracking system (SMART; Panlab S. I., Barcelona, Spain) was used.

Two-sample comparisons were carried out using the Student t-test, while multiple comparisons were made using one-way ANOVA followed by the Newman-Keuls multiple range test. All data were presented as the means±S.E.M. The statistical differences were accepted at the 5% level unless otherwise indicated.

### <10-1> Open Field Test

Locomotor activity was measured in the open field of a white Plexiglas chamber (45×45×45 cm). Illumination in the chamber was adjusted to 70 lux. The mice were all placed in the same environment as that of the chamber 30 minutes prior to the test. Each mouse was placed individually in the middle of the open field and locomotion was recorded for 60 minutes. The horizontal locomotor activity was judged according to the distance the animal moved. The inner 30 percentage of the open filed was defined as the center of the chamber.

As a result, the locomotor activities shown by Tg-βCTF99/B6 at 7-11 months were similar to those of wild type controls. The locomotor activity displayed by Tg-βCTF99/B6 at 14 month was slightly elevated, compared to that of the wild type controls, though it was not significant (FIG. 8A). The approaches to the center of the open field (a sign of anxiety) were also similar to those of control mice.

### <10-2> Rota-Rod Test

Rota-rod test was performed to evaluate motor coordination and motor learning. Rota-rod consists of a rotating cylinder (4.5 cm in diameter) with a speed controller attached. Mice were placed on the top of the cylinder where they have access to tight grip. Rota-rod was spinned at the speed of 5-20 rpm, and the speed was gradually increased. Cut-off time was set as 3 minutes and intertrial interval was 60 minutes. Hang-on time on rod was measured.

As a result, motor coordination of the transgenic mice of the present invention at 5.5 months was similar to that of wild type controls. However, motor coordination of the transgenic mice of the present invention at 11 months was reduced, compared to that of the wild type controls, though the difference was not significant (FIG. 8B).

### <10-3> Morris Water Maze Test

Morris water maze test is a hippocampus-dependent analysis method that depends largely on the capability of an animal to learn and remember the relation between stimulus at a distance and hidden platform for escape (Morris et al., 1982, Nature, 297, 701). That is, in this test, forced swimming or the latency to find a hidden platform by taking advantage of spatial indices memorized during placing on the platform was observed. Based on this observation, cognitive function of a mouse was investigated and quantified by comparison of the distance and the time that the mouse swam. In order to investigate memory retention of a mouse, the locations of the entrance to the pool and the hidden platform were changed often, while spatial indices were still located same. Particularly, water maze consisted of a 90 cm-diameter cylinder pool filled with 22° C. opaque milky water. A 10 cm-diameter hidden platform was placed in a quadrant 1.5 cm below the surface of the opaque water. The pool was placed in a room with abundant environmental and artificial cues including a window, a chair and posters. In the course of daily testing, mice were admitted successively into each of the quadrants and allowed to swim for 90 seconds maximum. On locating the platform, the animals were permitted to remain on it for 30 seconds before the session was terminated. The latency to find the platform for each of two trails and the average of the two trains were recorded for each mouse.

As a result, wild type controls at 7, 11 and 14 months could recognize the Indices of the hidden platform, and this achievement improved trial after trail. On the other hand, Tg-βCTF99/B6 mice at 7, 11 and 14 months showed longer latency to find the hidden platform, compared to the wild type controls, Indicating the cognitive deficits, even though the difference was not very significant (FIG. 9A-B). In the meantime, swimming speeds of Tg-βCTF99/B6 mice at 7, 11 and 14 months were similar to those of age-matched controls (FIG. 9C). Those results indicate that Tg-βCTF99/B6 mice show elevated cognitive deficits, compared to the wild type controls.

### <10-4> Passive Avoidance Test

Mice prefer darkness to lightness. When mice are allowed to choose one of the two chambers, one is lighted and the other is dark chamber, they have no hesitation to go for the dark chamber. Mice are once placed in a lighted chamber and then allowed to move to a dark chamber but a strong electric shock is given then (that is, a training). After the training, when mice are forced to select a chamber to enter, most of wild type mice try to stay in a lighted chamber without the electric shock, even though unwillingly. Passive avoidance test is designed based on the above idea, and so the test is to investigate learning and memory retention through spatial information such as a lighted and a dark chamber, and an electric shock.

Particularly, the test apparatus of the invention consisted of a brightly lit and a dark, compartment (15×15×15 cm each), each equipped with a shock-grid floor, and a door between the two chambers. During the first day of testing, each mouse was placed in the lighted chamber and left to habituate to the apparatus for 5 minutes, while allowing it to explore the light and dark rooms. On the second day, the mice were placed in the lighted chamber. After 30 seconds, the middle door was opened and the latency for the mouse to enter the dark chamber was measured. When the mouse entered the dark room, the door was closed and two successive electric foot-shocks (100 V, 0.3 mA, 2 seconds) were delivered through the grid-floor. After training, mice were individually replaced in the lighted chamber and the latency to enter the dark chamber was measured.

As a result, pre-shock latency to enter the dark chamber of wild type control mice at 7-14 months was similar to that of Tg-βCTF99/B6 mice of the present invention. However, the post-shock latency to enter the dark chamber of Tg-βCTF99/B6 mice of the present invention was much shorter than that of wild type control mice (FIG. 9D). The results indicate that the transgenic mice of the present invention show cognitive deficits.

### <10-5> Elevated Plus Maze Test

Increased anxiety is a problematic symptom of human AD patients (Folstein and Bylsma, 1999, Alzheimer Disease (Eds by Terry et al.,) 2nd. Lippincott Williams & Wilkins, Philadelphia). So, the present inventors needed to investigate the possibility of increased anxiety by the introduction of APP mutant gene in Tg-APP/B6 transgenic mice. Elevated plus maze apparatus consisted of four arms (30×7 cm) made of black Plexiglas, which were placed at right angles to each other and elevated 50 cm above the floor. Two of the arms had 20 cm high walls (enclosed arms), while other two had no walls (open arms). The illumination at the center was adjusted to 40 lux. For the test, the mouse was initially placed at the center of the platform and left to explore the arms for 5 minutes. The number of entries in the open and in the enclosed arms and the time spent in each arm was recorded. Entry into each arm was scored as an event if the animal placed all four paws into the corresponding arm.

As a result, the number of entries into open and enclosed arms for Tg-βCTF99/B6 at 7 months was similar to that of age-matched controls. However, the number of entries and the time spent in the open arm for the Tg-βCTF99/B6 transgenic mice at 13 months was less than that of age-matched controls. The results indicate that Tg-βCTF99/B6 mice of the present invention show increased anxiety (FIG. 10).

### INDUSTRIAL APPLICABILITY

As explained hereinbefore, unlike the wild type control mice, the transgenic mice of the present invention showed notable cognitive deficits, meaning the impaired memory retention, in Morris water maze test. In addition, in elevated plus maze test, the transgenic mice of the present invention showed increased anxiety. Those results confirmed that the transgenic mice of the present invention showed clinical symptoms of AD better than any other conventional AD animal models. The transgenic mice of the present invention showed age-dependent neuronal loss, which is superior to any known conventional AD animal models. Therefore, the transgenic mice of the present invention are expected to serve as a useful AD model for the study of AD-related pathogenesis including the study of cognitive deficits.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A vector, for transformation of animals to induce Alzheimer's disease pathology, that contains a gene coding a protein represented by SEQ. ID. No 10 containing C-terminal fragment (CTF) of mutant human amyloid beta precursor protein (APP) in which 698^{th} amino valine (V) of AP751 is replaced with phenylalanine (F).

2. The vector for transformation of animals to induce Alzheiner's disease pathology as set forth in claim 1, wherein the vector additionally includes a promoter and polyadenylation region.

3. The vector design for transformation of animals to induce Alzheimer's disease pathology as set forth in claim 2, wherein the promoter is human PDGF-β promoter.

4. The vector for transformation of animals to induce Alzheimer's disease pathology as set forth in claim 2, wherein the polyadenylation region is SV40 pA.

5. The vector for transformation of animals to induce Alzheimer's disease pathology as set forth in claim 2, wherein the vector additionally includes Kozac sequence between a promoter and a gene coding C-terminal fragment of the mutant human amyloid beta precursor protein.

6. The vector for transformation of animals to induce Alzheimer's disease pathology as set forth in claim 2, wherein the vector additionally includes nucleotide sequence coding signal peptide in front of a gene coding C-terminal fragment of mutant human amyloid beta precursor protein.

7. The vector for transformation of animals to induce Alzheimer's disease pathology as set forth in claim 6, wherein the nucleotide sequence is represented by SEQ. ID. No 25.

8. The vector for transformation of animals to induce Alzheimer's disease pathology as set forth in claim 2, wherein the vector is designed to include human PDGF-β promoter gene, mutant gene coding an amino acid sequence represented by SEQ. ID. No 3 and SV40 pA in that order, and represented by the cleavage map PDGF-βCTF99(V717F)-pA.

9. The vector for transformation of animals to induce Alzheimer's disease pathology as set forth in any of claim 2-claim 7, wherein the vector additionally includes intron between a promoter gene and a mutant gene coding a mutant protein.

10. The vector for transformation of animals to induce Alzheimer's disease pathology as set forth in claim 9, wherein the intron is intron B that is derived from human beta-globin gene.

11. The vector for transformation of animals to induce Alzheimer's disease pathology as set forth in claim 9, wherein the vector is designed to include human PDGF-β promoter gene, intron B gene of human beta-globin, mutant gene coding an amino acid sequence represented by SEQ. ID. No 3 and SV40 pA in that order, and represented by the cleavage map PDGF-intron-βCTF99(V717F)-pA.

12. A transgenic mouse with induced Alzheimer's disease pathology generated by introducing the vector for transformation of animals of claim 1.

13. The transgenic mouse with induced Alzheimer's disease pathology as set forth in claim 12, wherein the mouse is Tg-βCTF/B6 showed clinical symptoms of AD such as motor coordination deficit, impaired memory retention, cognitive deficits and increased anxiety (Accession No: KCTC 10609BP).
